SEQUENCE LISTING (10 sequences)

SEQ ID NO: 1 - protein, 262 aa, Homo sapiens
  polypeptide of spot 8 and 66
DLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSK

SEQ ID NO: 2 - protein, 377 aa, Homo sapiens
  polypeptide of spot 110
DLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIK

SEQ ID NO: 3 - protein, 274 aa, Homo sapiens
  polypeptide of spot 65
DLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEK

SEQ ID NO: 4 - protein, 110 aa, Homo sapiens
  polypeptide of spot 34
CCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 5 - protein, 194 aa, Homo sapiens
  polypeptide of spot 125
AIQLTYNPDESSKPNMIDAATLKSRKMLEEIMKYEASILTHDSSIRYLQEIYNSNNQKIVNLKEKVAQLEAQCQEPCKDTVQIHDITGKDCQDIANKGAKQSGLYFIKPLKANQQFLVYCEIDGSGNGWTVFQKRLDGSVDFKKNWIQYKEGFGHLSPTGTTEFWLGNEKIHLISTQSAIPYALRVELEDWNGR

SEQ ID NO: 6 - protein, 650 aa, Homo sapiens
  polypeptide of spot 182
WCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGYFAVAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVK

SEQ ID NO: 7 - protein, 327 aa, Homo sapiens
  polypeptide of spot 183
YAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGR

SEQ ID NO: 8 - protein, 393 aa, Homo sapiens
  polypeptide of spot 87
EIQNAVNGVKQIKTLIEKTNEERKTLLSNLEEAKKKKEDALNETRESETKLKELPGVCNETMMALWEECKPCLKQTCMKFYARVCRSGSGLVGRQLEEFLNQSSPFYFWMNGDRIDSLLENDRQQTHMLDVMQDHFSRASSIIDELFQDRFFTREPQDTYHYLPFSLPHRRPHFFFPKSRIVRSLMPFSPYEPLNFHAMFQPFLEMIHEAQQAMDIHFHSPAFQHPPTEFIREGDDDRTVCREIRHNSTGCLRMKDQCDKCREILSVDCSTNNPSQAKLRRELDESLQVAERLTRKYNELLKSYQWKMLNTSSLLEQLNEQFNWVSRLANLTQGEDQYYLRVTTVASHTSDSDVPSGVTEVVVKLFDSDPITVTVPVEVSRKNPKFMETVAEK

SEQ ID NO: 9 - protein, 319 aa, Homo sapiens
  polypeptide of spot 101
ENEGFTVTAEGKGQGTLSVVTMYHAKAKDQLTCNKFDLKVTIKPAPETEKRPQDAKNTMILEICTRYRGDQDATMSILDISMMTGFAPDTDDLKQLANGVDRYISKYELDKAFSDRNTLIIYLDKVSHSEDDCLAFKVHQYFNVELIQPGAVKVYAYYNLEESCTRFYHPEKEDGKLNKLCRDELCRCAEENCFIQKSDDKVTLEERLDKACEPGVDYVYKTRLVKVQLSNDFDEYIMAIEQTIKSGSDEVQVGQQRTFISPIKCREALKLEEKKHYLMWGLSSDFWGEKPNLSYIIGKDTWVEHWPEEDECQDEENQK

SEQ ID NO: 10 - protein, 1194 aa, Homo sapiens
  c3c polypeptide
SPMYSIITPNILRLESEETMVLEAHDAQGDVPVTVTVHDFPGKKLVLSSEKTVLTPATNHMGNVTFTIPANREFKSEKGRNKFVTVQATFGTQVVEKVVLVSLQSGYLFIQTDKTIYTPGSTVLYRIFTVNHKLLPVGRTVMVNIENPEGIPVKQDSLSSQNQLGVLPLSWDIPELVNMGQWKIRAYYENSPQQVFSTEFEVKEYVLPSFEVIVEPTEKFYYIYNEKGLEVTITARFLYGKKVEGTAFVIFGIQDGEQRISLPESLKRIPIEDGSGEVVLSRKVLLDGVQNPRAEDLVGKSLYVSATVILHSGSDMVQAERSGIPIVTSPYQIHFTKTPKYFKPGMPFDLMVFVTNPDGSPAYRVPVAVQGEDTVQSLTQGDGVAKLSINTHPSQKPLSITVRTKKQELSEAEQATRTMQALPYSTVGNSNNYLHLSVLRTELRPGETLNVNFLLRMDRAHEAKIRYYTYLIMNKGRLLKAGRQVREPGQDLVVLPLSITTDFIPSFRLVAYYTLIGASGQREVVADSVWVDVKDSCVGSLVVKSGQSEDRQPVPGQQMTLKIEGDHGARVVLVAVDKGVFVLNKKNKLTQSKIWDVVEKADIGCTPGSGKDYAGVFSDAGLTFTSSSGQQTAQRAELQCPQPAASNLDEDIIAEENIVSRSEFPESWLWNVEDLKEPPKNGISTKLMNIFLKDSITTWEILAVSMSDKKGICVADPFEVTVMQDFFIDLRLPYSVVRNEQVEIRAVLYNYRQNQELKVRVELLHNPAFCSLATTKRRHQQTVTIPPKSSLSVPYVIVPLKTGLQEVEVKAAVYHHFISDGVRKSLKVVPEGIRMNKTVAVRTLDPERLGRSEETKENEGFTVTAEGKGQGTLSVVTMYHAKAKDQLTCNKFDLKVTIKPAPETEKRPQDAKNTMILEICTRYRGDQDATMSILDISMMTGFAPDTDDLKQLANGVDRYISKYELDKAFSDRNTLIIYLDKVSHSEDDCLAFKVHQYFNVELIQPGAVKVYAYYNLEESCTRFYHPEKEDGKLNKLCRDELCRCAEENCFIQKSDDKVTLEERLDKACEPGVDYVYKTRLVKVQLSNDFDEYIMAIEQTIKSGSDEVQVGQQRTFISPIKCREALKLEEKKHYLMWGLSSDFWGEKPNLSYIIGKDTWVEHWPEEDECQDEENQKQCQDLGAFTESMVVFGCPN

(19) United States
(12) Patent Application Publication
(10) Pub. No.: US 2013/0196924 A1
(43) Pub. Date: Aug. 1, 2013

(54) BIOMARKERS FOR DIAGNOSING AND DETECTING THE PROGRESSION OF NEURODEGENERATIVE DISORDERS, IN PARTICULAR OF AMYOTROPHIC LATERAL SCLEROSIS

(71) Applicants: Enrico Maria Bucci, Ivrea (IT); Chiara Abrescia, Torino (IT); Alessandra Giuliano Albo, Torino (IT); Paolo Bongioanni, Pisa (IT); Massimo Natale, Pavone Canavese (IT); Davide Corpillo, Pecco (IT); Vincenzo De Tata, Pisa (IT); Lorenza Franciosi, Masserano (IT); Katarzyna Lis, Ivrea (IT)

(72) Inventors: Enrico Maria Bucci, Ivrea (IT); Chiara Abrescia, Torino (IT); Alessandra Giuliano Albo, Torino (IT); Paolo Bongioanni, Pisa (IT); Massimo Natale, Pavone Canavese (IT); Davide Corpillo, Pecco (IT); Vincenzo De Tata, Pisa (IT); Lorenza Franciosi, Masserano (IT); Katarzyna Lis, Ivrea (IT)

(73) Assignee: Bioindustry Park Silvano Fumero S.p.A., Colleretto Giacosa (IT)

(21) Appl. No.: 13/131,223

(22) PCT Filed: Nov. 25, 2009
(86) PCT No.: PCT/IB09/07580

§ 371 Date: May 25, 2011

(30) Foreign Application Priority Data
Nov. 25, 2008 (IT) TO2008A000870

(51) Int. Cl. G01N 33/68

(52) U.S. Cl. CPC G01N 33/6896
USPC 514/17.7; 530/350; 506/9; 436/501; 435/7.92; 506/18

(57) ABSTRACT

The present invention relates to biomarkers, to their use and to a method for diagnosing in vitro or detecting the progression of a neurodegenerative disease in an individual, in particular for Amyotrophic Lateral Sclerosis (ALS). The method comprises the steps of isolating a biological sample from the individual; quantifying the level of one or more polypeptides in the biological sample according to the invention; comparing the obtained level with a reference level.

### TECHNICAL FIELD

The present invention relates to biomarkers for diagnosing and detecting the progression of neurodegenerative disorders, in particular of amyotrophic lateral sclerosis (also referred hereinafter as ALS).

### STATE OF THE ART

Amyotrophic Lateral Sclerosis (ALS), also known as Lou Gehrig's disease, is the most severe motor neuron disease: it is a devastating disorder of the central nervous system (CNS) with a multifactor aetiopathogenesis and a lethal course. ALS affects 5 in every 100,000 individuals each year [Julien, 2001], and is therefore the third most common cause of death in adults due to neurodegenerative diseases, after Alzheimer's disease (AD) and Parkinson's disease [source: Motor Neuron Disease Association].

There are both familial and sporadic forms of ALS. Familial ALS accounts for 5-10% of all cases and has been correlated to several genetic mutations: about 20% of familial cases are associated to mutations in the gene for superoxide dismutase 1 (SOD1), an ubiquitously expressed antioxidant protein. Familial ALS and sporadic ALS are clinically undistinguishable, suggesting a common pathogenesis, possibly triggered by heterogeneous molecular events [Bruijn et al., 2004].

From an anatomopathological point of view, ALS is characterized by a rapid and selective loss of motor neurons in the brain, brainstem and/or spinal cord, negatively affecting the strength, growth and function of muscles. Early symptoms of ALS are quite heterogeneous and may include arm, leg and trunk weakness, spasticity, breathing difficulty, mastication and deglutition difficulty, and slurred speech.

Progressive atrophy of skeletal muscles and paralysis follow, the most common cause of lethality being respiratory failure generally within 3 to 5 years from the clinical onset [Belsh, 1996; Rowland, 1998].

There is currently no effective therapy for ALS. The only approved drug, riluzole (a compound with anti-glutamatergic activity), slightly prolongs survival and partially relieves symptoms, but has no effect on blocking the progression of neurodegeneration, regressing the symptoms and partially recovering motor functions [Lacomblez et al., 1996; Miller et al., 2002].

Biomedical research on ALS has, among its priorities, the discovery of biomarkers for the disease, as currently there are no clinical tools for the molecular diagnosis of ALS. As a matter of fact, although there are many ongoing studies, publications and patent applications on the subject, there are currently no ALS-specific biomarkers available for clinical use [Shaw e Williams, 2000; Bowser et al., 2006]. In particular, no biomarker has proven capable of discriminating ALS patients from individuals with non-neurological inflammatory processes, nor are there disease progression markers available. In the absence of biochemical parameters allowing to diagnose ALS at an early stage, diagnostics is based only on the clinical observation of the symptoms which are often serious and invalidating and presumably appear well after the related pathogenic molecular events have already occurred. This may be one of the reasons why the available treatments only have a limited effect. It should also be noted that, in the absence of effective therapies, an early diagnose would allow to immediately start a therapy and therefore prolong life expectancy of the patient [Lacomblez et al., 1996; Miller et al., 2002].

The absence of biomarkers also does not allow an efficient classification on molecular bases of the different phenotypes of ALS, a multifactorial and complex disease which probably includes different subtypes within its clinical definition [Shaw e Williams, 2000; Bowser et al., 2006].

The absence of specific biomarkers on the other side reflects the poor knowledge of the molecular mechanisms involved in the onset and development of ALS, with the consequence that a quantitative indication of the efficacy may not be drawn for compounds screened for therapy. The discovery of new biomarkers may therefore contribute to the understanding of the mechanisms of the disease, and thus to the development of possible new therapeutic targets.

### DISCLOSURE OF INVENTION

In view of the above, the need for new specific ALS biomarkers results apparent. It is an object of the present invention to therefore provide polypeptides which may be used as biomarkers, in particular for diagnosing and detecting the progression of ALS.

According to the present invention this object is achieved by means of polypeptides according to claims 1 and 2. The present invention indeed includes the use of polypeptides of claims 1 and 2 for diagnosing neurodegenerative diseases, in particular for diagnosing ALS.

According to the present invention there is also provided the use of polypeptides according to claims 1 and 2 for detecting the progression of ALS.

### DEFINITIONS

Unless otherwise explicitly specified, the following terms have the following meaning.

In the present disclosure “identity percentage” and “% identity” between two sequences of amino acids (peptides) or nucleic acids (nucleotides) means the percentage of amino acid residues or identical nucleotides at corresponding positions in the two aligned sequences when ideally aligned.

To determine the “identity percentage” of two amino acid or nucleic acid sequences, the sequences are aligned with one another; to achieve an optimum comparison, gaps (i.e. deletions or insertions—which may possibly even be arranged at the ends of the sequences) may be introduced in the sequences. The amino acid and nucleotide residues at corresponding positions are therefore compared. When a position in the first sequence is occupied by the same amino acid or nucleotide residue that occupies the corresponding position in the second sequence, the molecules are identical in this position. The identity percentage between two sequences is a function of the number of identical positions shared by the sequences [i.e. identity=(number of identical positions/total number of positions)×100].

According to an advantageous embodiment, the sequences have the same length.

Advantageously, the compared sequences do not have any gaps (or insertions).

The identity percentage may be obtained by using mathematical algorithms. A non-limitative example of a mathematical algorithm used for the comparison of two sequences is the Karlin and Altschul algorithm [Proc. Natl. Acad. Sci. USA 87 (1990) 2264-2268] modified by Karlin and Altschul [Proc. Natl. Acad. Sci. USA 90 (1993) 5873-5877]. Such an algorithm is incorporated in the BLASTn and BLASTp software by Altschul [Altschul, et al, J. Mol. Biol. 215 (1990) 403-410].

In order to obtain alignments also in the presence of one or more gaps (or insertions) methods may be used which give a relatively high penalty to each gap (or insertion) and a lower penalty for each additional amino acid or nucleotide residue in the gap (such an additional amino acid or nucleotide residue is defined as an extension of the gap). High penalties will obviously determine optimum alignments with a lower number of gaps.

An example of a software adapted to carry out this kind of alignment is the BLAST software as disclosed in Altschul, et al., Nucleic Acids Res. 25 (1997) 3389-3402. For this purpose the BLASTn and BLASTp software may be used with default parameters. A BLOSUM62 matrix is usually employed with BLAST softwares.

An advantageous and non-limitative example of a software to carry out an optimum alignment is GCG Wisconsin Bestfit package (Wisconsin University, USA; Devereux et al., 1984, Nucleic Acids Research 12:387). Default parameters which provide a penalty of −12 for a gap and a penalty of −4 for each extension for an amino acid sequence are also used in this case.

In the present disclosure “homology percentage” and “% homology” between two amino acid or nucleic acid sequences means the percentage of homologous amino acid or nucleotide residues at corresponding positions in the two sequences when ideally aligned.

The homology percentage between two sequences is determined in substantially the same manner as described above for the determination of the identity percentage except that homologous positions and not only identical positions are considered in the computation.

As far as nucleotide sequences are concerned, two homologous positions have two nucleotides which are different but lead to the same amino acid.

As far as amino acid sequences are concerned, two homologous positions have two homologous amino acids, i.e. amino acids having similar chemical-physical properties, for instance amino acids belonging to the same groups such as: aromatic (Phe, Trp, Tyr), acid (Glu, Asp), polar (Gln, Asn), basic (Lys, Arg, His), aliphatic (Ala, Leu, Ile, Val), with a hydroxy-group (Ser, Thr), with a short side chain (Gly, Ala, Ser, Thr, Met). Substitutions among these homologous amino acids are not expected to change the phenotype of the proteins (conservative amino acid substitutions). Specific examples of conservative substitutions are known in this technical field and are disclosed in literature (for example, Bowie et al., Science, 247:1306-1310 (1990)).

Further examples of software and/or items related to the determination of alignments and of homology and/or identity percentages are indicated for example in US2008003202, US2007093443, WO06048777.

In the present text, “corresponding position” means a position in an amino acid or nucleotide sequence corresponding (facing), upon alignment, to a determined position of a reference sequence.

### BRIEF DESCRIPTION OF THE FIGURES

For a better understanding of the present invention, the invention is now also described with reference to the accompanying figures, in which:

FIG. 1A is a table listing the spots corresponding to the biomarkers for diagnosing ALS identified by means of two-dimensional electrophoresis (2DE), and the respective SEQ ID NO, Accession number, number of peptides of the Mascot identification, covered sequence, apparent isoelectrical point (pI), apparent molecular weight (MW), Mowse score of the Mascot identification (MS score);

FIG. 1B is a table listing the spot corresponding to the biomarker for detecting the progression of ALS identified by 2DE, and the respective SEQ ID NO, Accession number, number of peptides, covered sequence, apparent pI, apparent PM, MS score;

FIG. 2 represents a two-dimensional gel in which the spots listed in the table of FIG. 1A are highlighted;

FIG. 3 is a table showing, for each of the spots identified by 2DE, the average values of the percentage volumes (% Vol) of each group of individual tested plasma samples (i.e. group of ALS patients, group of cardiovascular patients (INF) and group of healthy controls (CTR)), obtained as disclosed in the following, the standard deviations corresponding to the average % Vol within the group, and the values of statistic p expressing the significance of each spot in distinguishing the ALS group with respect to each of two reference groups on the basis of the % Vol;

FIG. 4A is a diagrammatic representation of the three-dimensional magnification of spot 8 detected with Coomassie staining, as disclosed in the following;

FIG. 4B is an image of spot 8 on pools of plasma samples from healthy controls (A) and ALS patients (B). In the top panels, spot 8 (indicated by an arrow) is shown by Coomassie staining, while in the bottom panels it is shown by 2DE Western blotting;

FIG. 5 is a graph showing the levels of the biomarker corresponding to spot 8 in individuals analysed in the form of % Vol;

FIG. 6 is a histogram of the values of the diagnostic function DF, computed as disclosed in the following, for each of the individuals included in the study;

FIG. 7A is a graph which shows the trend of the % Vol of spot 101 in 10 ALS patients subjected to riluzole therapy, comparing two samples taken at an interval of 10-12 months for each patient;

FIG. 7B is a graph which shows the trend of the % Vol of spot 101 in 8 ALS patients subjected to riluzole+lithium therapy, comparing two samples taken at an interval of 3-5 months for each patient;

FIG. 7C is an image of spot 101 shown by Western Blotting on 2 pools of plasma samples—Time 1 and Time 2—taken at an interval of 10-12 months.

FIG. 8 shows the minimum amino acid sequences of the albumin protein fragments corresponding to spot 65 (SEQ ID NO: 3, 110 (SEQ ID NO: 2), 8 (SEQ ID NO: 1), 66 (SEQ ID NO: 1), 34 (SEQ ID NO: 4), aligned with respect to the amino acid sequence of complete albumin (Accession number: 28592); and

FIG. 9 contains the image of a two-dimensional gel showing spot 101 (listed in the table of FIG. 1B).

### DETAILED DESCRIPTION OF THE INVENTION

The identification of novel biomarkers was carried out by screening plasma proteins. The passage of molecules from the CNS to plasma allows the direct identification in the plasma of biomarkers coming from disease sites, which are added to the possible alteration of specific plasma markers. Furthermore, the sampling of peripheral fluids allows for the collection of a greater number of samples during the discovery step of the biomarkers, providing a greater statistical robustness of the results obtained.

A two-dimensional electrophoresis (2DE) screening of the plasma proteome of patients suffering from ALS was carried out comparing the latter with healthy individuals and non-neurological patients sampled shortly after a heart-stroke or coronary-stroke (in the following referred to as cardiovascular patients).

The concept at the basis of applied proteome research is that most diseases result in a variation in the amount of proteins and peptides in the body fluids and tissues. Proteomic strategies may reveal the disruption of the balance in the distribution of different protein isoforms or modifications in proteins such as those deriving from oxidative stress, directly or indirectly related to the aetiological mechanisms [Perluigi et al., 2005]: For this reason much of the current applied biochemical research is directed to the study of the proteome of biological fluids to identify biomarkers related to different pathological conditions. As regards complex biological mixtures such as blood, different proteomic analyses have already identified proteins specifically associated to non-genetic diseases and unrelated to tumours [Aivado et al., 2007; Kim et al., 2007; Li et al., 2007; Avasarala et al., 2005].

It should be noted that not always are the identified biomarkers rare proteins or fragments thereof, or proteins or parts of proteins the expression of which is peculiar and rare. On the contrary, many publications report disease biomarkers which consist of modified forms of common and ubiquitary proteins such as albumin and proteins related thereto [Yagame et al., 1995; Kaiser et al., 2004; Funding et al., 2005; German et al., 2007]. An important advantage of proteomics based on 2DE consists in the possibility of identifying alterations in the amount of fragments and modified forms of proteins for which the level of the intact molecule does not significantly change [Finehout et al., 2007]. Peculiar protein fragments specific for ALS and other diseases with a strong apoptotic component, may derive from the selective activation of proteases and specific degradation pathways, which are usually active at low levels [Ilzecka et al., 2001]. In particular, as far as neurodegenerative diseases are concerned, it has been shown that C-terminal fragments of albumin present in the cerebrospinal fluid (CSF) of patients with AD are specific disease biomarkers, probably because the disease implies alterations in the degradation process of albumin [Finehout et al., 2007]. It has also been shown that albumin fragments have toxic activity on organotypic cultures of cholinergic neurons and on primary cultures of astrocytes [Moser and Humpel, 2007]. Therefore, peptides deriving from the degradation of albumin or other abundant plasma proteins could represent not only an epiphenomenon of the disease, but also of the main players in the pathogenesis.

For the proteomic study that has led to the identification of the biomarkers of the disease, the plasma proteome of three groups of individuals was considered:
Healthy subjects (controls, n=14)
Cardiovascular patients, within 6 hours of heart-stroke or coronary-stroke (n=8)
ALS patients (n=27)

The cardiovascular patients were included in the study as a “filter” to eliminate the biomarkers that discriminate ALS patients from controls only due to the inflammatory process ongoing in the patients. The plasma proteome of two groups of ALS patients was considered for the proteomic study that led to the identification of the progression biomarker: A study was first carried out on 10 patients subjected to a treatment with riluzole (analysing two samples—T1 and T2—for each patient performed at an interval of 10-12 months). The result was then confirmed by a second study on 8 patients subjected to a treatment with riluzole+lithium (analysing two samples—T0-lithium and T1-lithium—for each patient performed at an interval of 3-5 months). Fresh blood from all patients was sampled by standard methods with their informed consent.

The plasma samples were then centrifuged at 400 g for 10′ at 4° C. Albumin was not depleted from the sample, not only because albumin is a carrier protein which may bind to interesting markers, but also because (as previously disclosed) modified forms of albumin differentially present in the disease at issue may have a diagnostic role. The depletion of albumin may therefore lead to loss of useful biomarkers [Kawakami et al., 2005; German et al., 2007].

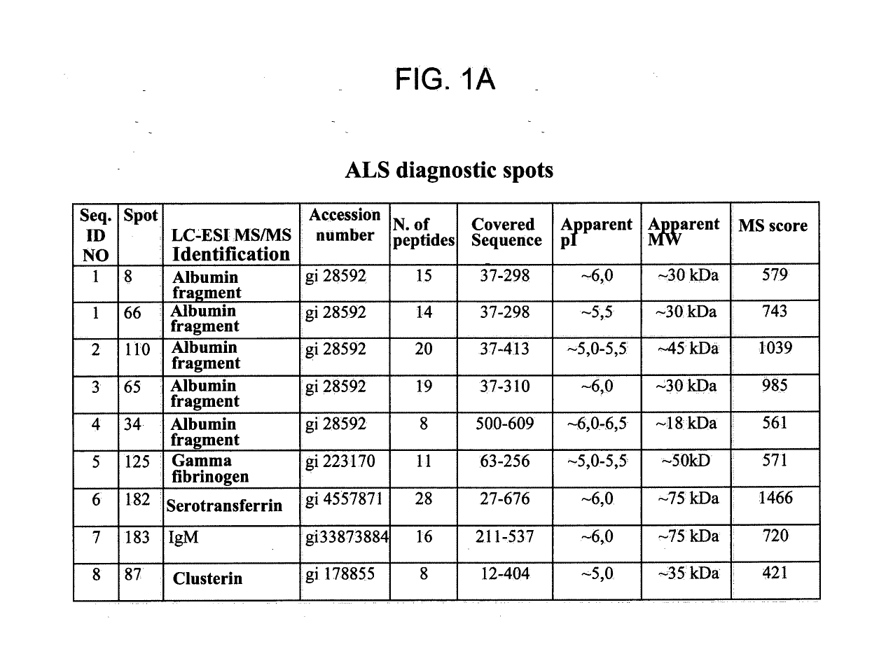

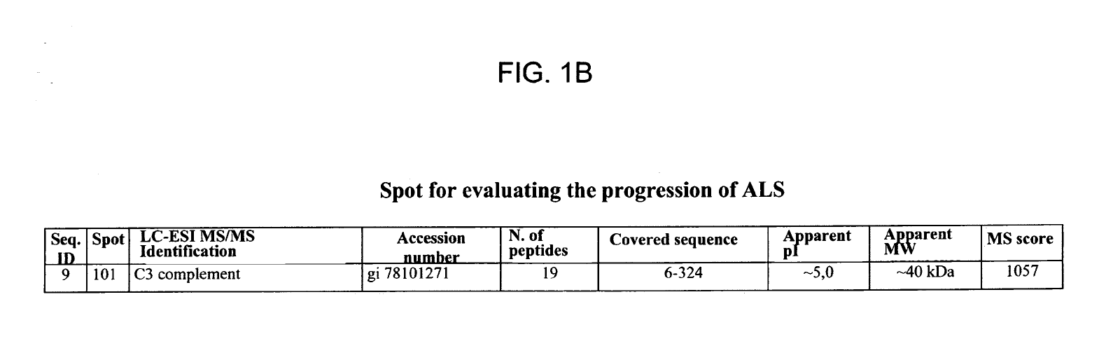

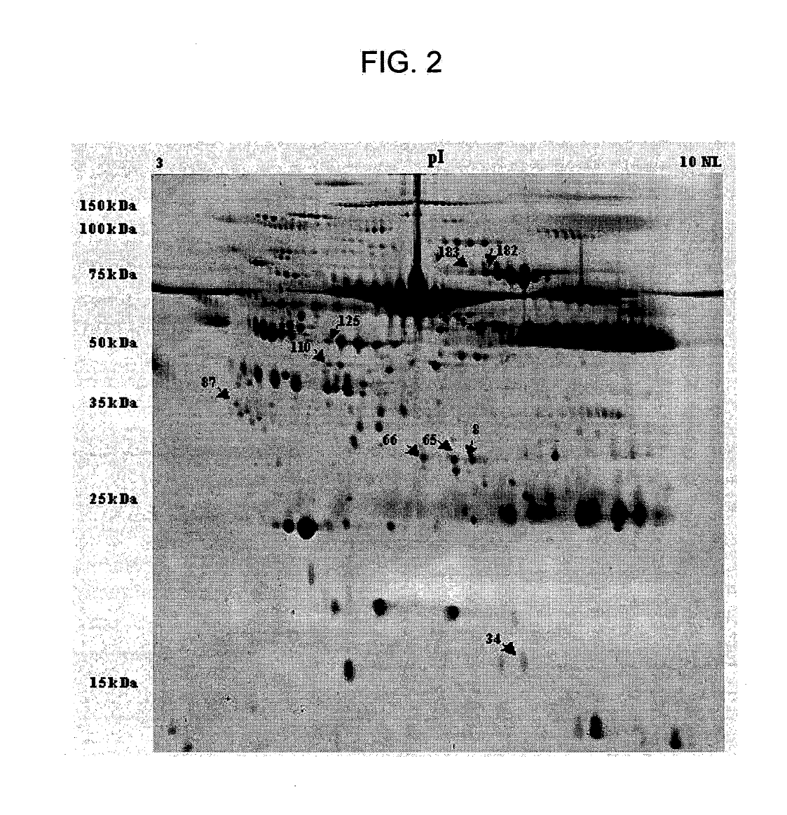

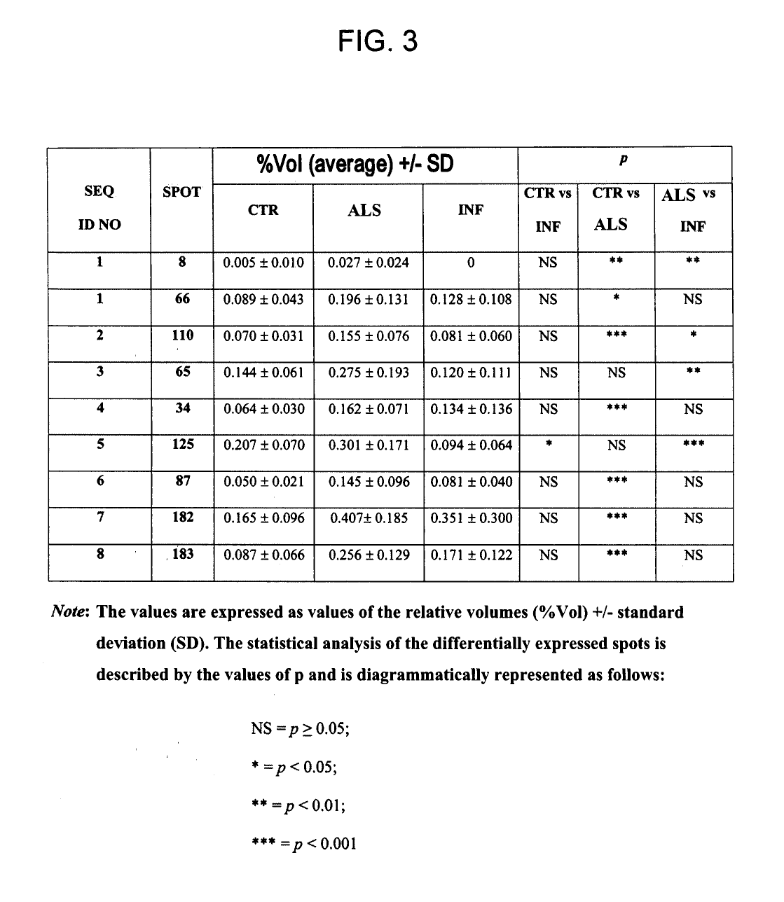

Once the samples were prepared, 2DE was performed for each sample and for at least one technical replicate according to Jacobs et al. [2001] with some modifications. In brief, 6 μl (about 400 μg of total protein in case of Coomassie staining), or 1.5 μl (about 100 μg of total protein in case of Sypro staining) of each plasma sample were heated for 5′ at 95° C. with 10 μl of SDS 5% w/v, DTT 2.5% w/v, and diluted to 330 μl with 7 M urea, 2 M thiourea, 4% w/v CHAPS, 0.5% v/v of IPG buffer 3-10 NL (GE Healthcare, Uppsala, Sweden), and traces of bromophenol blue, as in Hughes et al., 1992. The sample was then loaded on cm IPG 3-10 NL strips (GE Healthcare, Uppsala, Sweden) for isoelectrofocusing (IEF) by in-gel rehydration (2 h at 0 V, 12 h at 30 V). IEF was then performed at 20° C. on an IPGphor apparatus (GE Healthcare) as follows: 500 V at 500 V/hr, 1,000 V at 1,000 V/hr with a linear gradient; 8,000 V at 13,500 V/hr with a linear gradient, 8,000 V at 72,000 V/hr. Prior to SDS-PAGE (SDS-polyacrylamide gel denaturing electrophoresis), the IPG strips were equilibrated twice for 15′ in a buffer containing 50 mM Tris-HCl pH 8.8, 6 M urea, 30% v/v glycerol, 2% w/v SDS, and traces of bromophenol blue, containing 1% w/v DTT for the first equilibration step and 2.5% w/v iodoacetamide for the second one. SDS-PAGE was performed on 12.5% polyacrylamide gels (1.5 mm thick) according to Laemmli [1970] but without stacking gel, using a Hoefer SE600 apparatus (GE Healthcare). The second dimension was run at 60 mA/gel at 16° C. until the bromophenol blue dye front reached the bottom of the gel. Proteins with a molecular weight (MW) in the range between 15 and 100 kDa and with an isoelectric point (pI) in the range between 4.5 and 8.5 were used as standards for the calibration of the MWs and of the pls. The gels were coloured with Coomassie brilliant blue R350 (Sigma, San Diego, US) or with Sypro Ruby (Molecular Probes, Invitrogen). Stained gels were scanned with an ImageMaster Labscan V3.0 scanner (GE Healthcare) for the gels stained with Coomassie blue or with a CCD camera (Perkin Elmer) for the gels coloured with Sypro. Images were analysed with the ImageMaster 2-DE Platinum 5.0 software (GE Healthcare). For each group, a reference gel was chosen, i.e. the gel containing the highest amount of well focalized spots. Spots were detected and gels in turn matched with their respective reference. Spots present in more than 70% of the gels in a given group were used to create a synthetic gel (average gel) that represents the average proteome of each group. The three average gels were subsequently matched to detect the spots shared by the three examined populations; the subsequent quantitative analysis consisted in comparing the corresponding percentage volumes (% Vol) of each of these spots in the three populations. For each spot, the % Vol was calculated as an integral of the volume of each spot stained with Coomassie blue (area of the spot multiplied by its intensity) normalised by the sum of the volumes of all of the spots in the reference average gel. Spots, which consistently and significantly varied among the three populations, were extracted through a non-parametric ANOVA (Kruskal-Wallis test), plus a multiple comparisons post-test (Dunn's test), and p values<0.05 were considered statistically significant. The spots identified in this manner are listed in FIG. 1A and shown in FIG. 2 (spot 65, 66, 110, 125, 182, 183, 87, 34, i.e. SEQ ID NO: 1-8, excluding spot 8). The average % Vol values and the standard deviations for each of the spots are summarised in FIG. 3, highlighting the differences among the ALS patients and the rest of the individuals considered. Spot 110 allows to distinguish ALS patients both from controls and from cardiovascular patients. The experimental variability among technical replicates of the same sample has been determined by comparing the different % Vol obtained, and did not result in any case above 0.8 times (average variability: 0.45 times; minimum variability: 0.2 times).

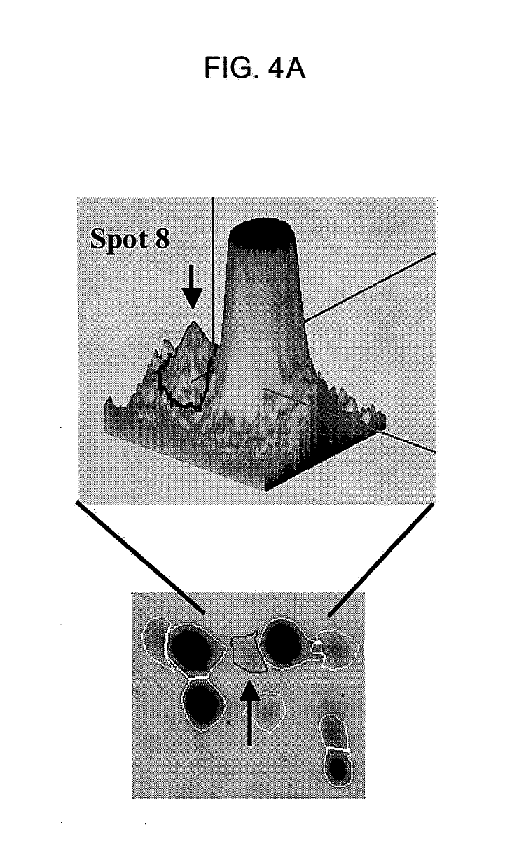

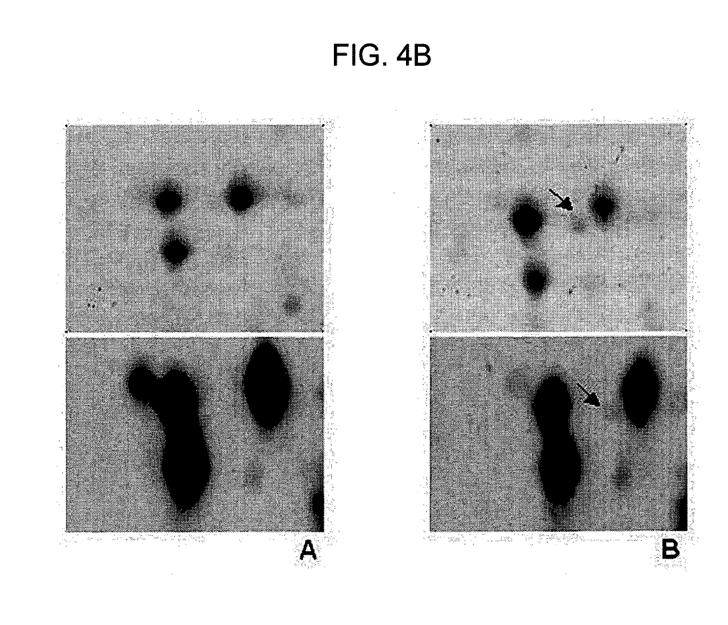

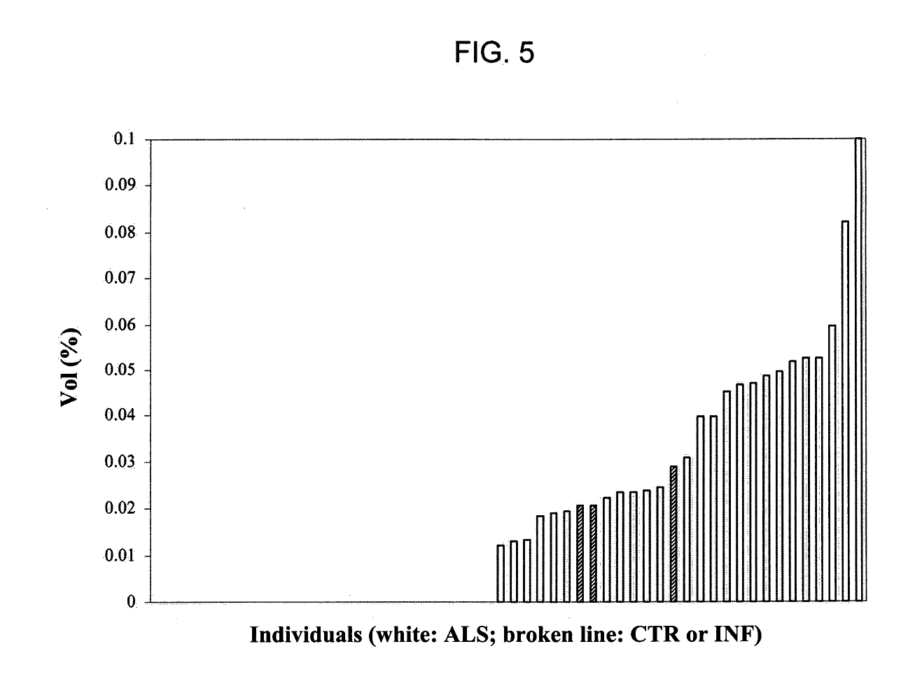

After the quantitative analysis was completed in the manner disclosed above, unmatched spots were subjected to a qualitative analysis, in order to identify those proteins or protein fragments representative of the ALS patient group only, and absent in the other groups, or vice-versa (qualitative analysis). Spot 8 (SEQ ID NO: 1) is also an identified ALS marker (FIG. 1A, FIG. 2; the three-dimensional magnification of spot 8 stained with Coomassie blue appears in FIG. 4A; the levels of this marker in the tested individuals are shown in FIG. 5).

Considering all of the 9 spots, a post-test discriminating analysis has been performed to assay the ability thereof to classify each individual in the appropriate population, on the basis of the measured % Vol (software: StatistiXL). The overall correct prediction efficiency reaches 89.8% with the correct identification of all of the ALS patients and of 81.5% of ALS patients. The same analysis has led to the formulation of the following linear function (Diagnostic Function, DF):

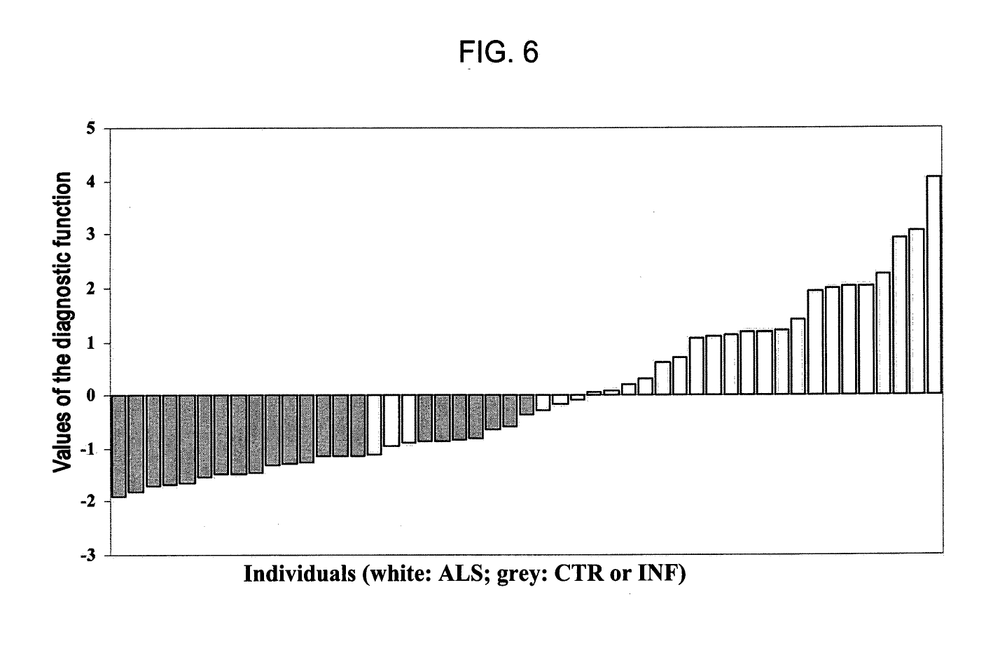

DF=−3.349% Vol(182)+8.688% Vol(183)−1.146% Vol(65)+5.536% Vol(110)+1.652% Vol(87)+2.630% Vol(125)+3.026% Vol(34)−2.426% Vol(66)+30.098% Vol(8)−2.38

in which % Vol(n) indicates the % Vol of spot n, according to the numbering indicated on the 2DE map of FIG. 2. The correspondence between the numbering of the spots considered for the computation of the DF as shown in FIG. 2 and of their respective SEQ ID NOs, used elsewhere in the present patent, has been shown in FIG. 1A. The value of the DF for each of the subjects included in the study is shown in FIG. 6. A positive value of this function is clearly a diagnostic parameter for ALS, while a negative value tends to be associated to individuals not suffering from ALS.

The average values of the DF, the corresponding standard deviations and the size of the considered groups have been taken as input values for the following

Power Analysis, comprising the computation of Cohen's D parameter, which accounts for the effect of the size of the sample in the determination of the normalised size effect, as disclosed by Cohen [1992] and by Hedges and Olkin [1985]. The Power Analysis which was performed therefore provides a measure of the confidence by which the same size effect may be observed on the entire population.

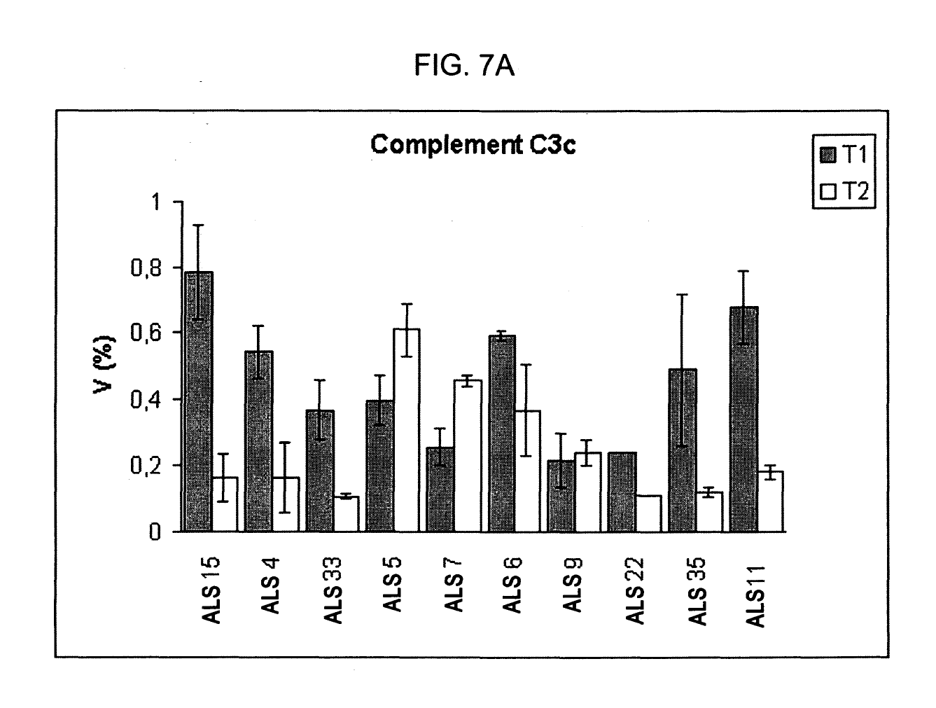

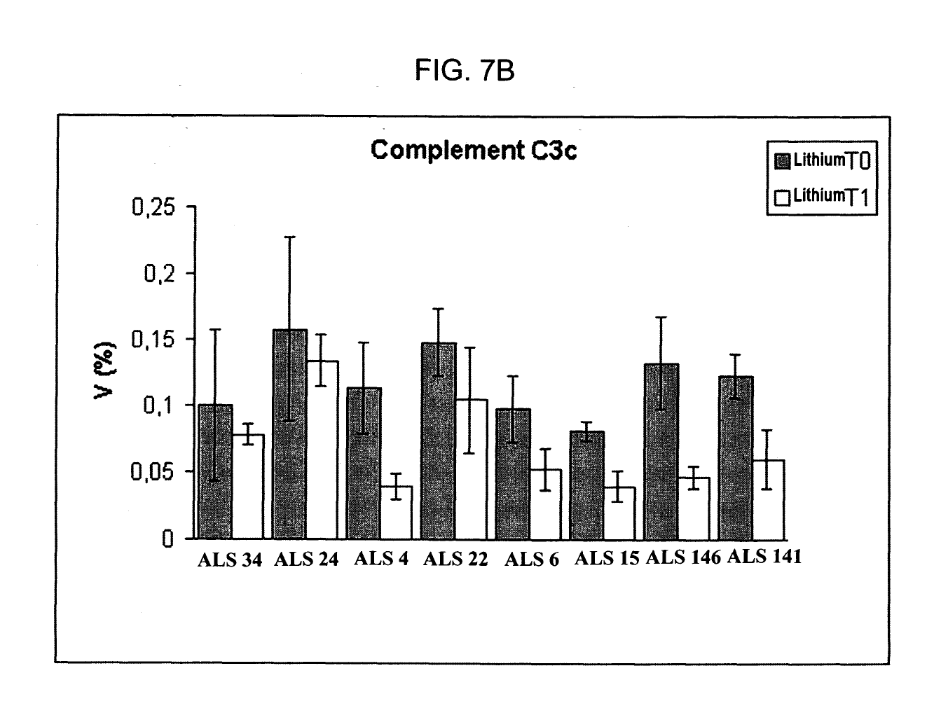

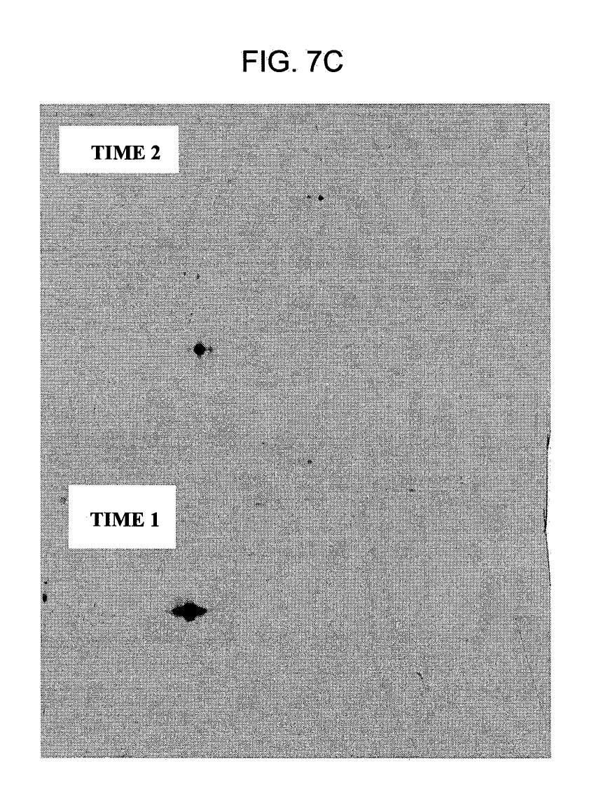

As regards the disease progression biomarkers, a non-parametric analysis (Mann-Whitney test) was carried out with a threshold of p≦0.05 in order to identify the spots which vary consistently and significantly between the two populations of samples (T1 vs T2, T0-lithium vs T1-lithium), as shown in FIGS. 7A and 7B: Spot 101 is an ALS progression biomarker.

Once the biomarkers were identified in the disclosed manner, the identity thereof was determined by mass spectrometry. In particular, the selected protein spots were excised manually from the gels and destained overnight with 40% ethanol in 25 mM ammonium bicarbonate, washed twice with 25 mM ammonium bicarbonate, three times with acetonitrile, and dried. Each gel fragment was rehydrated in 25 mM ammonium bicarbonate containing 0.6 μg of modified porcine trypsin and digested overnight at 37° C. Peptides were extracted by sonication in 25 mM ammonium bicarbonate, loaded onto a ZORBAX 300 SB C18 RP column (75 μm×150 mm, 3.5 μm particles, Agilent, Santa Clara, Calif., USA) and eluted with a gradient of acetonitrile from 5% to 80% (containing 0.1% formic acid) at a flow rate of 0.3 μl/min by an HP 1100 nanoLC system coupled to a XCT-Plus nanospray-ion trap mass spectrometer (Agilent) (elsewhere referred to as LC-ESI MS/MS. MS parameters were the following: scan range m/z=100-2,200, scan speed 8,100 m/z s-1, dry gas flow 5 l/min, dry temperature 300° C., capillary 1.8 kV, skimmer 40 V, ion charge control (ICC) target 125,000, maximum accumulation time 300 ms. Positively charged peptides were automatically isolated and fragmented, and spectra were deconvoluted by the DataAnalysis software version 3.4 (Bruker Daltonics, Bremen, Germany). Mass spectrometry data obtained by LC-ESI MS/MS were fed to the Mascot search algorithm for searching against the NCBI non-redundant database (http://www.matrixscience.com—mass tolerance for the monoisotopic peak masses was set to 1.8 Da for the parent ion or 0.8 Da for the fragments; the maximum number of non-cut sites per peptide was 3). Allowed modifications were cysteine carbamidomethylation and methionine oxidation. Hits with a probability-based Mowse score higher than 47 were considered significant (p<0.05).

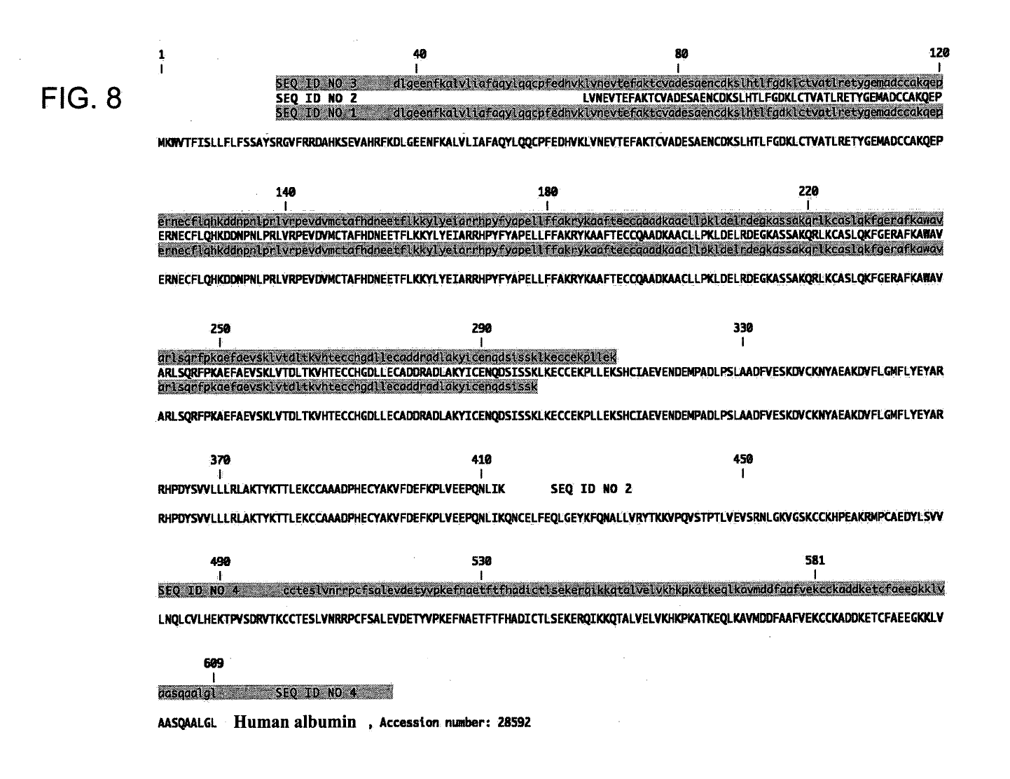

The protein identity of the biomarkers obtained in this manner is shown in FIG. 1 and indicated with the corresponding SEQ ID NOs. As clearly results, albumin fragments (SEQ ID NO:1-4) are predominant. 1-4). The minimum amino acid sequence of these fragments is shown in FIG. 1A and diagrammatically shown in FIG. 8 as compared to the whole albumin sequence, identified by Accession Number 28592, which results being the reference sequence for human serum albumin. Fragments of this sequence have been identified in the present study as the biomarkers which are the object of the present patent.

“Minimum fragment sequence” means the sequence obtained by examining the peptides deriving from the tryptic digestion of a fragment, by sorting the peptides on the basis of the sequence of the native protein, and obtaining the sequence included between the N-terminal amino acid of the first peptide and the C-terminal amino acid thereof. This sequence is included within the fragment at issue, but is not limited thereto. Further amino acids may be present both at the N-terminal and at the C-terminal included between the identified tryptic sites and the following tryptic site (in an N or C-terminal direction). Therefore, although the fragments corresponding to spot 66 and spot 8 in FIG. 1A (highlighted in the two-dimensional map of FIG. 2) have the same minimum sequence (SEQ ID NO: 1), they are probably different at the C-terminal (as both minimum sequences start at the N-terminal end of mature albumin). In particular, as there are many acidic residues immediately downstream of the minimum sequence, it is possible that the fragment corresponding to spot 66 has some C-terminal acidic residues more than the fragment corresponding to spot 8, in accordance with its more acidic isoelectrical point (FIG. 2, FIG. 8).

The fragmentation of albumin in vivo is altered in many conditions, among which the after-effects of hematopoietic stem cell transplant [Kaiser et al., 2004], exocrine pancreatic damage [Walgren et al., 2007], acute corneal rejection [Funding et al., 2005], meningococcal sepsis [Holland et al., 2001], diabetic nephropathy [Yagame et al., 1995], ischemic heart disease, acute inflammation, endotoxicosis and ageing [Bito et al., 2005]. As regards the CNS and neurodegenerative diseases, specific albumin fragments have already been reported as being biomarkers: for instance, some C-terminal fragments of albumin present in the CSF represent specific biomarkers for AD [Finehout et al., 2007]. Furthermore, a specific serum albumin fragment has been found to be increased 2.8 times in a mouse model of muscle dystrophy [Doran et al., 2006]. As ALS is characterised by an extensive activation of serum proteases [Ilzecka et al., 2001; Demestre et al., 2006] and by a strong oxidative stress [Barber et al., 2006], it is feasible that some degradation mechanisms typical of the disease produce specific albumin fragments, such as those included in the set of biomarkers disclosed in FIG. 1A and shown in FIG. 8 against the sequence of the whole albumin.

Another biomarker which was identified and corresponds to spot 182 (FIG. 1A and FIG. 2; SEQ ID NO: 6), is a glycoform of transferrin. It should be recalled that, in ALS patients, transferrin accumulates in Bunina bodies [Mizuno et al., 2006], that the SOD1 protein modulates the expression of the transferrin receptor [Danzeisen et al., 2006], and finally that defects in the expression of alsin cause the intracellular accumulation of transferrin in motoneuron cultures [Jacquier et al., 2006]. If singularly considered, and not in combination with the other biomarkers disclosed in the present invention, transferrin would have a limited value as an ALS biomarker, as transferrin glycoforms are involved in an aspecific manner in different neurodegenerative and non-neurodegenerative diseases [Zeman et al., 2000; Brettschneider et al., 2008]. However, we have verified that the inclusion of these two spots in the identified set of biomarkers increases the overall diagnostic power of the set, probably because, as recalled, there are some mechanisms which lead to the alteration of transferrin in ALS patients.

Another biomarker that was identified and corresponds to spot 183 (SEQ ID NO: 7) is the constant chain of IgMs. The involvement of IgMs in ALS is well documented and mainly based on serological evidence on patients. High anti-GM1 ganglioside IgM titres are commonly found in patients with peripheral neuropathies and neuromotory syndromes [Pestronk, 1991]. More recently, high titres of IgMs against GM2 and GD2 were also dosed [Mizutani et al., 2003]. Noteworthy, IgM is the isotype of serum immune responses reported against neurofilament proteins [Couratier et al., 1998]. Therefore, in general, the IgM fragment identified as an ALS marker could derive from the IgM related immune response, reported in more than one study on ALS patients.

Another biomarker identified with the disclosed method is chain A of gamma-fibrinogen (spot 125, SEQ ID NO: 5). Although fibrinogen is not synthesised in the CNS, the increase of fibrinogen gamma A chain in the CSF is thought to be connected to blood-CSF barrier damage and fibrinogen is generally regarded as a marker of inflammation associated to neurological diseases, since it is known that the nervous system is especially able to produce fibrin receptors and fibrin-dependent intracellular signalling molecules under inflammatory conditions [discussed in Akassoglou e Strickland, 2002]. Recent studies have shown that macrophages in the CNS and Schwann cells in the peripheral nervous system are the two cytotypes most commonly involved in phenomena correlated with extravasation of fibrinogen and products resulting from the degradation thereof. Impairment of the fibrinolysis pathway is closely associated to the pathogenesis of MS, for which neuroinflammation is one of the main pathological features [Adams et al., 2004]. Furthermore, in the CSF of AD patients, an increase in chain A of gamma-fibrinogen has a role as disease biomarker, although this increase may be merely due to damage of the hematoencephalic barrier [Lee et al., 2007].

The last ALS biomarker that was identified (spot 87; SEQ ID NO: 8) was found to be a form of clusterin (Apo J). An increase in the mRNA for clusterin was shown by in situ hybridisation in areas of active neurodegeneration of the spinal cord [Grewal et al., 1999]. Clusterin may have a complex role in neurodegenerative processes: as well as having an inhibiting activity on the cell membrane anchoring complex, this multifunctional glycoprotein may promote cell aggregation and serve as molecular chaperone, preventing the aggregation of denatured proteins. The increase of the mRNA level of clusterin and of the protein itself may be detected in cerebral ischemic damage and in many neurological diseases, among which AD, multiple sclerosis and epilepsy. In some cells, the induction of the expression of clusterin is associated to apoptosis; non-neural cells engineered so as to produce reduced amounts of clusterin are more sensitive to oxidative stress [Grewal et al., 1999].

Among these 9 biomarkers, spot 8 has proved especially efficient:
it allows to discriminate the population of ALS patients from both control populations (healthy and cardiovascular patients);
the different expression of spot 8 in ALS patients with respect to healthy controls has also been tested by 2DE Western blotting (FIG. 4B).

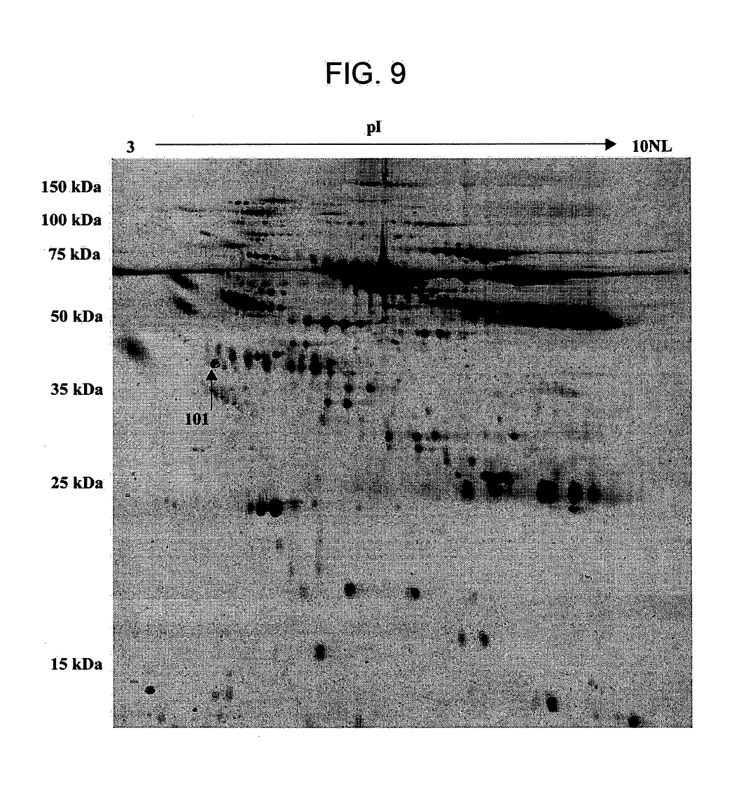

As regards the ALS progression biomarker, the analysis by LC-ESI MS/MS has identified it as complement component 3, in particular fragment 2 of chain α′ of C3c (SEQ ID NO: 9). In non-denaturing conditions, this fragment is bound by disulphide bonds to other 2 polypeptide chains again deriving from complement C3: chain β′ and fragment 2 of chain α′ (polypeptide c3c SEQ ID NO: 10) (http://www.uniprot.org/). C3c fragments have already been identified by 2DE as peripheral ALS markers [Goldknopf et al., 2006]: however the fragments reported in Goldknopf et al. do not correspond to the specific fragment disclosed in the present invention, as is apparent from the totally different pI (and therefore from the different position in the 2DE maps obtained from serum or plasma, an example of which is shown in FIG. 9). Furthermore, C3c has never been involved in the progression of the disease, and therefore the C3c fragment corresponding to spot 101 represents a truly new ALS progression biomarker.

It should be clear at this point to the person skilled in the art that any combination of disclosed biomarkers, with different statistical power, may be used for the differential diagnosis of ALS with respect to other neurodegenerative diseases, as well as for its progression. Furthermore, each combination of such biomarkers may be used together with other biomarkers to obtain a better predictive and statistical power. For example, the disclosed biomarkers, and in particular the % Vol evaluated by 2DE, may be used in combination with the ALS serum markers discovered by Goldknopf et al. [2006], both for the diagnosis and for the evaluation of the stage of progression of the disease. It is apparent that such combinations fall within the aim and scope of the present patent, as do other combinations of other types of biomarkers and/or physiological and/or diagnostic markers.

### DESCRIPTION OF ONE OR MORE EMBODIMENTS

The diagnostic procedure object of the present invention may be carried out by different embodiments.

By way of mere example, the following paragraph discloses an embodiment based on the sampling of blood from subjects to be tested, on the quantification by 2DE of the identified biomarkers, on the computation of a diagnostic function to identify the presence of ALS and on the evaluation of the stage of progression of the disease by comparing the amount of C3c on 2DE between diachronic samples.

As disclosed in the paragraph directed to the variants of the suggested method, it should be understood that the procedure disclosed in detail in the following is one among the many possible procedures which exploit the same set of biomarkers, which procedures must be considered, as a whole, as falling within the spirit and the scope of the present patent.

In particular, the present invention is based on the discovery of a set of ALS biomarkers, the amount of which is correlated with the presence of the disease (all of the biomarkers indicated in FIG. 1A, i.e. all except C3c) or with its progression (only C3c, FIG. 1B). The amount of these biomarkers may be evaluated by 2DE, as previously disclosed, but it is apparent to a person skilled in the art that any other evaluation method of the level of one or more of the biomarkers shown in FIG. 1 falls within the scope and spirit of the present patent application. By mere way of example, the biomarkers may be quantified by one or more of the following alternative techniques:
1. Western blot
2. Enzyme-Linked ImmunoadSorbent Assay (ELISA)
3. High Pressure Liquid Chromatography (HPLC)
4. Mass spectrometry

Furthermore, a variant falling within the scope of the present patent application consists in using any numerical combination of the amount of some or all the disclosed biomarkers to compute a different diagnostic (linear or non-linear) function or derive any statistical parameter so as to obtain a score useful for the diagnosis of ALS or for the evaluation of its progression.

It should also be understood that any combination of the present biomarkers with other diagnostic methods for ALS or other neurological disorders must be considered to be part of the present patent application.

It should finally be understood that, although the use of human blood samples is preferable as compared to other biological material, the testing and use of any combination of biomarkers shown in FIG. 1 in biological samples other than human blood must be part of the present patent application.

Diagnostic Procedure

In order to diagnose ALS in an individual, or evaluate the stage of progression of the disease, the following paragraphs disclose:

(1) a method for quantifying the biomarkers object of the present patent;

(2) a method for diagnosing ALS based on the quantification of previous item (1);

(3) a method for evaluating the stage of progression of ALS based on the quantification of the C3c biomarker obtained with the procedure of item (1).

(1). Quantification of the Biomarkers

The plasma of the individuals involved was obtained by standard methods. Once the plasma samples were prepared (by centrifugation at 4° C. for 10′ at 400 g), a 2DE experiment was carried out for each sample and for the corresponding technical replicate, according to Jacobs et al. [2001] with some modifications. In brief, 6 μl (about 400 μg of total protein, in case of Coomassie staining), or 1.5 μl (about 100 μg of total protein, in case of Sypro staining) of each plasma sample were heated for 5′ at 95° C. with 10 μl of SDS 5% w/v, DTT 2.5% w/v, and diluted to 330 μl with 7 M urea, 2 M thiourea, 4% w/v CHAPS, 0.5% v/v of IPG buffer 3-10 NL, and traces of bromophenol blue, as in Hughes et al., 1992. The sample was then loaded on 18 cm IPG 3-10 NL strips by in-gel rehydration (2 h at 0 V, 12 h at 30 V). Isoelectrofocusing was then performed at 20° C. on an IPGphor apparatus (GE Healthcare) or equivalent as follows: 500 V at 500 V/hr, 1,000 V at 1,000 V/hr with a linear gradient; 8,000 V at 13,500 V/hr with a linear gradient, 8,000 V at 72,000 V/hr. Prior to SDS-PAGE, the IPG strips were equilibrated twice for 15′ in a buffer containing 50 mM Tris-HCl pH 8.8, 6 M urea, 30% v/v glycerol, 2% w/v SDS, and traces of bromophenol blue containing 1% w/v DTT for the first equilibration step, and 2.5% w/v iodoacetamide for the second one. SDS-PAGE was performed on 12.5% polyacrylamide gels (1.5 mm thick) according to Laemmli [1970] but without stacking gel, using a Hoefer SE600 apparatus (GE Healthcare) or equivalent apparatus. The second dimension was run at 60 mA/gel at 16° C. until the bromophenol blue dye front reached the bottom of the gel. Standard proteins having MW (15-100 kDa) and pI (pH 4.5-8.5) may be used for the calibration of the MW and of the pI. The gels must then be stained with Coomassie brilliant blue R350 (Sigma) or with Sypro Ruby (Molecular Probes, Invitrogen). After staining, the digital images of the gels are acquired by using an ImageMaster Labscan V3.0 scanner (GE Healthcare) or an equivalent for the gels stained with Coomassie or a CCD camera (Perkin Elmer) for gel stained with Sypro, and the images are analysed with the ImageMaster 2-DE Platinum 5.0 software (GE Healthcare) or an equivalent. To identify the diagnostic spots on the tested gel, the image of the gel is overlapped with the appropriate reference gel (FIG. 2). The % Vol is obtained for each diagnostic spot by densitometric analysis as a percentage ratio of the normalised density of the spot on the total of the density of all of the spots aligned between the examined gel and the reference gel.

To carry out the 2DE Western blot experiments, the plasma proteins were denatured and subsequently separated on a 2DE gel, as disclosed for Coomassie and Sypro staining. After the gels were run, they were immediately introduced into an aqueous solution containing 25 mM Tris, 40 mM 6-aminohexanoic acid and 20% v/v methanol, checking that the final pH was 9.4. The proteins separated thereby were transferred to a nitrocellulose membrane (Hybond C-extra with 0.45 micrometre pores; GE Healthcare, Uppsala, Sweden) by applying a “semi-dry” transfer. After transfer, the membranes were incubated for 1 hour at 42° C. in a blocking solution containing TBS and 0.1% w/v Tween 20 (T-TBS) and 3% w/v fish gelatine. T-TBS was also used for washing away unspecific antibody binding. A polyclonal ALB(N17) Santa Cruz antibody was used as a primary recognition antibody at a dilution of 1:500. As the antibody is raised in goat, an anti-goat HRP (horse radish peroxidase) conjugate was used as secondary detection antibody; the membrane was therefore incubated with a specific chemiluminescent substrate provided by the ECL Western Blotting kit (Pierce, Euroclone). The images corresponding to the proteins identified after film exposure, were acquired by an ImageMaster Labscan V3.0 (GE Healthcare, Uppsala, Sweden).

2) ALS Diagnosis

(2.1). Computation

The following diagnostic function DF may be computed from the % Vol of the spots identified in FIG. 1:

DF=−3.349% Vol(182)+8.688% Vol(183)−1.146% Vol(65)+5.536% Vol(110)+1.652% Vol(87)+2.630% Vol(125)+3.026% Vol(34)−2.426% Vol(66)+30.098% Vol(8)−2.38

wherein it should be understood that the numbering of each spot shown highlighted by way of example on the 2DE map in FIG. 1 corresponds to the SEQ ID NO shown in FIG. 1A for each spot, i.e.:

Spot 8 and spot 66=SEQ ID NO 1;

spot 110=SEQ ID NO 2;

spot 65=SEQ ID NO 3;

spot 34=SEQ ID NO 4;

spot 125=SEQ ID NO 5;

spot 182=SEQ ID NO 6;

spot 183=SEQ ID NO 7;

spot 87=SEQ ID NO 8;

It should also be understood that any other function (for example a different linear function or a non-linear function) of the indicated amounts of biomarkers obtained as disclosed or by Western Blot, ELISA or other methods, may be used instead of the disclosed function, and falls within the scope of the present patent.

(2.2). Evaluation of the Results

As shown in FIG. 6, the value of DF tends to be positive in the presence of ALS. It is therefore assumed that, in case the quantification of the suggested biomarkers leads to a value of DF>0, the tested individual suffers from ALS.

The person skilled in the art will have no difficulty in recognising that, if a different function is used, a different threshold value must be selected, but the information inputted, however related to one or more of the reported biomarkers, is the same and is covered by the present patent.

(3) Progression of the Disease

(3.1). Computation

To identify spot 101 on the test gel, the same gel is overlapped to the image of a reference gel (FIG. 9). The % Vol of spot 101 is therefore computed as previously disclosed.

(3.2). Evaluation

In the course of time from the clinical onset of the disease, the % Vol of spot 101 is expected to decrease in ALS patients (FIGS. 7A, 7B and 7C). Therefore, a comparison of the % Vol of this spot with the corresponding value obtained in a previous moment is informative of the progression of the disease. As is apparent to the person skilled in the art, the measurement of the amount of protein corresponding to spot 101 (C3c) by any other means may replace the evaluation of the % Vol of spot 101, without departing from the scope of the present patent. The decrease of % Vol of spot 101 with the progression of the disease has also been studied by 2DE-Western blotting as disclosed hereinafter (FIG. 7C).

To carry out the 2DE Western blot experiments, the plasma proteins were denatured and subsequently separated on a 2DE gel, as disclosed for Coomassie and Sypro staining. After the gels were run, they were immediately introduced into an aqueous solution containing 25 mM Tris, 40 mM 6-aminohexanoic acid and 20% v/v methanol, checking that the final pH was 9.4. The proteins separated thereby were transferred to a nitrocellulose membrane (Hybond C-extra with 0.45 micrometre pores; GE Healthcare, Uppsala, sweden) by applying a “semi-dry” transfer. After transfer, the membranes were incubated for 1 hour at 42° C. in a blocking solution containing TBS and 0.1% w/v Tween 20 (T-TBS) and 5% w/v milk. T-TBS was used for washing away unspecific antibody binding. A polyclonal antibody (A 0062, DAKO) was used as a primary recognition antibody at a dilution of 1:5000. As it was raised in rabbit, an anti-rabbit HRP (horse radish peroxidase) conjugate was used as secondary detection antibody; the membrane was then incubated with a specific chemiluminescent substrate provided by the ECL Western Blotting kit (Pierce, Euroclone). The images corresponding to the proteins identified after film exposure were acquired by an ImageMaster Labscan V3.0 (GE Healthcare, Uppsala, Sweden).

### ADVANTAGES

The person skilled in the art and dealing with ALS will immediately recognise the advantages of a diagnostic test such as that disclosed and of the corresponding biomarkers, which have the following advantages:

1. greater objectivity with respect to clinical diagnostic methods, the test being related to a molecular aspect of the disease and to the measurement of quantitative parameters for the diagnosis;

2. greater accuracy of the suggested biomarkers with respect to others, as they are selected by considering two control groups, the first formed by healthy subjects and the second by cardiovascular subjects, to distinguish between specific ALS markers and generic inflammation markers;

3. simple sampling required for diagnosis, as the measurement is based on haematic biomarkers, small volumes of blood and on a single value for the diagnosis of ALS;

4. possibility of developing simplified diagnostic methodologies, as the detected biomarkers may be detected with techniques other than 2DE;

5. possibility of a follow-up at a quantitative level of the disease and of the therapies, as one of the detected biomarkers varies its level during the course of the disease.

Variants

What has been disclosed up to this point is a privileged example of the invention with some possible variations. The terms, descriptions and figures are shown by mere way of illustration and do not imply limitations in the aims or object of the present patent. The person skilled in the art will recognise that many possible variants are possible in the spirit and scope of the present invention, in the description of which each term has been used in the broadest sense possible, without any limitation unless explicitly indicated.

In particular, the present invention is based on the discovery of a set of ALS biomarkers, the amount of which is correlated to the presence of the disease (all of the biomarkers indicated in FIG. 1A, i.e. all except C3c) or to its progression (only C3c, FIG. 1B). The amount of these biomarkers may be evaluated by 2DE, as previously disclosed, but it is clear to a person skilled in the art that any other evaluation method of the level of one or more of the biomarkers shown in FIG. 1 falls within the scope and spirit of the present patent application. By mere way of example, the biomarkers may be quantified by one or more of the following alternative techniques:
1. Western blot
2. ELISA
3. HPLC
4. Mass spectrometry.

Furthermore, a variant falling within the scope of the present patent application consists in using any numerical combination of the amount of some or all of the disclosed biomarkers to compute a different diagnostic (linear or non-linear) function or derive any statistical parameter that provides a score useful for the diagnosis of ALS or for the evaluation of its progression.

It should also be understood that any combination of the present biomarkers with other diagnostic methods for ALS or other neurological disorders must be considered to be part of the present patent application.

It should finally be understood that, although the use of human blood samples is preferable as compared to other biological material, the testing and use of any combination of biomarkers shown in FIG. 1 in biological samples other than human blood must be part of the present patent application.

### REFERENCES

Adams R A, Passino M, Sachs B D, Nuriel T, Akassoglou K. (2004). Fibrin mechanisms and functions in nervous system pathology. Mol Interv, 4:163.
Aivado M, Spentzos D, Germing U, Alterovitz G, Meng X Y, Grall F, Giagounidis A A, Klement G, Steidl U, Otu H H, Czibere A, Prall W C, Iking-Konert C, Shayne M, Ramoni M F, Gattermann N, Haas R, Mitsiades C S, Fung E T, Libermann T A. (2007). Serum proteome profiling detects myelodysplastic syndromes and identifies CXC chemokine ligands 4 and 7 as markers for advanced disease. Proc Natl Acad Sci USA, 104:1307.
Akassoglou K e Strickland S. (2002). Nervous system pathology: the fibrin perspective. Biol Chem, 383:37.
Avasarala J R, Wall M R, Wolfe G M. (2005). A distinctive molecular signature of multiple sclerosis derived from MALDI-TOF/MS and serum proteomic pattern analysis: detection of three biomarkers. J Mol Neurosci, 25:119.
Barber S C, Mead R J, Shaw P J. (2006). Oxidative stress in ALS: a mechanism of neurodegeneration and a therapeutic target. Biochimica et Biophysica Acta, 1762:1051.
Belsh J M. (1996). Epidemiology and Historical Perspective of ALS. In: Belsh J M, Schiffmann P L (eds) Amyotrophic Lateral Sclerosis—Diagnosis and Management for the Clinician. Futura Publishing Company, 3.
Bito R, Hino S, Baba A, Tanaka M, Watabe H, Kawabata H. (2005). Degradation of oxidative stress-induced denatured albumin in rat liver endothelial cells. Am J Physiol Cell Physiol, 289:C531.
Bowser R, Cudkowicz M, Kaddurah-Daouk R. (2006). Biomarkers for amyotrophic lateral sclerosis. Expert Rev Mol Diagn, 6:387.
Brettschneider J, Mogel H, Lehmensiek V, Ahlert T, Süssmuth S, Ludolph A C, Tumani H. (2008). Proteome analysis of cerebrospinal fluid in amyotrophic lateral sclerosis (ALS). Neurochem Res, 2008 May 15. [Epub ahead of print]
Bruijn L I, Miller T M, Cleveland D W. (2004). Unraveling the mechanisms involved in motor neuron degeneration in ALS. Annu Rev Neurosci, 27: 723.
Cohen J. (1992). A power primer. Psychological Bulletin, 112:155.
Couratier P, Yi F H, Preud'homme J L, Clavelou P, White A, Sindou P, Vallat J M, Jauberteau M O (1998). Serum autoantibodies to neurofilament proteins in sporadic amyotrophic lateral sclerosis. J Neurol Sci, 154:137.
Danzeisen R, Achsel T, Bederke U, Cozzolino M, Crosio C, Ferri A, Frenzel M, Gralla E B, Huber L, Ludolph A, Nencini M, Rotilio G, Valentine J S, Carri M T. (2006). Superoxide dismutase 1 modulates expression of transferrin receptor. J Biol Inorg Chem, 11:489.
Demestre M, Howard R S, Orrell R W, Pullen A H. (2006). Serine proteases purified from sera of patients with amyotrophic lateral sclerosis (ALS) induce contrasting cytopathology in murine motoneurones to IgG. Neuropathol Appl Neurobiol, 32:141.
Doran P, Martin G, Dowling P, Jockusch H, Ohlendieck K. (2006). Proteome analysis of the dystrophin-deficient MDX diaphragm reveals a drastic increase in the heat shock protein cvHSP. Proteomics, 6:4610.
Finehout E J, Franck Z, Choe L H, Relkin N, Lee K H. (2007). Cerebrospinal fluid proteomic biomarkers for Alzheimer's disease. Ann Neurol, 61:120.
Funding M, Vorum H, Honore B, Nexo E, Ehlers N. (2005). Proteomic analysis of aqueous humour from patients with acute corneal rejection. Acta Ophthalmol Scand, 83:31.
German D C, Gurnani P, Nandi A, Garner H R, Fisher W, Diaz-Arrastia R, O'Suilleabhain P, Rosenblatt K P. (2007). Serum biomarkers for Alzheimer's disease: proteomic discovery. Biomed Pharmacother, 61383.
Goldknopf I L, Sheta E A, Bryson J, Folsom B, Wilson C, Duty J, Yen A A, Appel S H. (2006). Complement C3c and related protein biomarkers in amyotrophic lateral sclerosis and Parkinson's disease. Biochem Biophys Res Commun, 342:1034.
Grewal R P, Morgan T E, Finch C E. (1999). C1qB and clusterin mRNA increase in association with neurodegeneration in sporadic amyotrophic lateral sclerosis. Neurosci Lett, 271:65.
Hedges L e Olkin I, editori. (1985). Statistical Methods for Meta-Analysis. New York: Academic Press.
Holland P C, Hancock S W, Hodge D, Thompson D, Shires S, Evans S. (2001). Degradation of albumin in meningococcal sepsis. Lancet, 357:2102.
Hughes G J, Frutiger S, Paquet N, Ravier F, Pasquali C, Sanchez J C, James R, Tissot J D, Bjellqvist B, Hochstrasser D F. (1992). Plasma protein map: an update by microsequencing. Electrophoresis, 13:707.
Ilzecka J, Stelmasiak Z, Dobosz B. (2001). Interleukin-1beta converting enzyme/Caspase-1 (ICE/Caspase-1) and soluble APO-1/Fas/CD 95 receptor in amyotrophic lateral sclerosis patients. Acta Neurol Scand, 103:255.
Jacobs D I, van Rijssen M S, van der Heijden R, Verpoorte R. (2001). Sequential solubilization of proteins precipitated with trichloroacetic acid in acetone from cultured Catharanhus roseus cells yields 52% more spots after two-dimensional electrophoresis. Proteomics, 1:1345.
Jacquier A, Buhler E, Schafer M K, Bohl D, Blanchard S, Beclin C, Haase G. (2006). Alsin/Rac1 signaling controls survival and growth of spinal motoneurons. Ann Neurol, 60:105.
Julien J P. (2001). Amyotrophic lateral sclerosis: unfolding the toxicity of the misfolded. Cell, 104:581.
Kaiser T, Kamal H, Rank A, Kolb H J, Holler E, Ganser A, Hertenstein B, Mischak H, Weissinger E M. (2004). Proteomics applied to the clinical follow-up of patients after allogeneic hematopoietic stem cell transplantation. Blood, 104:340.
Kawakami T, Hoshida Y, Kanai F, Tanaka Y, Tateishi K, Ikenoue T, Obi S, Sato S, Teratani T, Shiina S, Kawabe T, Suzuki T, Hatano N, Taniguchi H, Omata M. (2005). Proteomic analysis of sera from hepatocellular carcinoma patients after radiofrequency ablation treatment. Proteomics, 5:4287.
Kim H J, Cho E H, Yoo J H, Kim P K, Shin J S, Kim M R, Kim C W. (2007). Proteome analysis of serum from type 2 diabetics with nephropathy. J Proteome Res, 6:735.
Lacomblez L, Bensimon G, Leigh P N, Guillet P, Meininger V. (1996). Dose-ranging study of riluzole in amyotrophic lateral sclerosis. Amyotrophic Lateral Sclerosis/Riluzole Study Group II. Lancet, 347:1425.
Laemmli U K. (1970). Cleavage of structural proteins during the assembly of the head of bacteriophage T4. Nature, 227:680.
Lee J W, Namkoong H, Kim H K, Kim S, Hwang D W, Na H R, Ha S A, Kim J R, Kim J W. (2007). Fibrinogen gamma-A chain precursor in CSF: a candidate biomarker for Alzheimer's disease. BMC Neurol, 7:14.
Li S Q, Yun J, Xue F B, Bai C Q, Yang S G, Que H P, Zhao X, Wu Z, Wang Y, Liu S J. (2007). Comparative proteome analysis of serum from acute pulmonary embolism rat model for biomarker discovery. J Proteome Res, 6:150.
Miller R G, Mitchell J D, Lyon M, Moore D H. (2002). Riluzole for amyotrophic lateral sclerosis (ALS)/motor neuron disease (MND). Cochrane Database Syst Rev, 2:CD001447.
Mizuno Y, Amari M, Takatama M, Aizawa H, Mihara B, Okamoto K. (2006). Transferrin localizes in Bunina bodies in amyotrophic lateral sclerosis. Acta Neuropathol, 112:597.
Mizutani K, Oka N, Kusunoki S, Kaji R, Kanda M, Akiguchi I, Shibasaki H. (2003). Amyotrophic lateral sclerosis with IgM antibody against gangliosides GM2 and GD2. Intern Med, 42:277.
Moser K V e Humpel C. (2007). Blood-derived serum albumin contributes to neurodegeneration via astroglial stress fiber formation. Pharmacology, 80:286.
Perluigi M, Poon H F, Hensley K, Pierce W M, Klein J B, Calabrese V, De Marco C, Butterfield D A. (2005). Proteomic analysis of 4-hydroxy-2-nonenal-modified proteins in G93A-SOD1 transgenic mice-A model of familial amyotrophic lateral sclerosis. Free Radic Biol Med, 38:960.
Pestronk A. (1991). Motor neuropathies, motor neuron disorders, and antiglycolipid antibodies. Muscle Nerve, 14:927.
Rowland L P. (1998). Diagnosis of amyotrophic lateral sclerosis. J Neurol Sci, 160:S6.
Shaw P J e Williams R. (2000). Serum and cerebrospinal fluid biochemical markers of ALS. Amyotroph Lateral Scler Other Motor Neuron Disord, 1:S61.
Walgren J L, Mitchell N D, Whiteley L O, Thompson D C. (2007). Evaluation of two novel peptide safety markers for exocrine pancreatic toxicity. Toxicol Sci, 96:184.
Yagame M, Suzuki D, Jinde K, Yano N, Naka R, Abe Y, Nomoto Y, Sakai H, Suzuki H, Ohashi Y. (1995). Urinary albumin fragments as a new clinical parameter for the early detection of diabetic nephropathy. Intern Med, 34:463.
Zeman D, Adam P, Kalistová H, Sobek O, Kelbich P, Andel J, Andel M. (2000). Transferrin in patients with multiple sclerosis: a comparison among various subgroups of multiple sclerosis patients. Acta Neurol Scand, 101:89.

1. A polypeptide comprising SEQ ID NO: 1, SEQ ID NO: 2, SEQ ID NO: 9, or SEQ ID NO: 10.

2. A polypeptide having a sequence with east 90% identity with SEQ ID NO: 1, SEQ ID NO: 2, SEQ ID NO: 9, or SEQ ID NO: 10.

3. A polypeptide having SEQ ID NO: 1, SEQ ID NO: 2, SEQ ID NO: 9, SEQ ID NO: 10.

4. A pharmaceutical composition comprising a polypeptide according to claim 1.

5. The composition according to claim 4 as a biomarker for diagnosing in vitro or detecting the progression of a neurodegenerative disorder in an individual.

6. Use of the polypeptide according to claim 1 or of the pharmaceutical composition according to claim 4 as a biomarker.

7. Use according to claim 6, for diagnosing in vitro a neurodegenerative disorder in an individual.

8. Use according to claim 6, for detecting the progression of a neurodegenerative disorder in an individual.

9. Use according to claim 6, wherein said neurodegenerative disorder is amyotrophic lateral sclerosis.

10. Use according to claim 6, wherein said biomarker is used in combination with at least another biomarker.

11. A method for diagnosing in vitro or detecting the progression of a neurodegenerative disorder in an individual comprising the steps of:
isolating a biological sample from an individual;
quantifying the level of one or more polypeptides according to claim 1 or a composition according to claim 4 in said biological sample;
comparing said level with a reference level.

12. The method according to claim 11, wherein said neurodegenerative disorder is amyotrophic lateral sclerosis.

13. The method according to claim 11, wherein said organic sample is a biological fluid.

14. The method according to claim 13, wherein said biological fluid is selected from the group consisting of blood, plasma, serum and cerebrospinal fluid.

15. The method according to claim 14, wherein the quantification of one or more polypeptides according to claim 1 or of a composition according to claim 4 is performed by means of a technique selected from the group consisting of two-dimensional electrophoresis, densitometry, Western blotting, ELISA, HPLC, mass spectrometry and protein chips.
